(19) United States
(12) Patent Application Publication
(10) Pub. No.: US 2003/0133960 A1
(43) Pub. Date: Jul. 17, 2003

(54) Cosmetic and dermatological uses of a retinoid and/or a carotenoid and acexamic acid

(75) Inventors: Albert Duranton, Maison Laffite (FR); Beatrice Renault, Saint Maurice (FR); Yann Mahe, Morsang Sur Orge (FR); Catherine Marion, Antony (FR); Philippe Catroux, Nogent Sur Marne (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'Oreal, Paris (FR)

(21) Appl. No.: 10/304,968

(22) Filed: Nov. 27, 2002

Related U.S. Application Data
(60) Provisional application No. 60/350,011, filed on Jan. 23, 2002.

(30) Foreign Application Priority Data
Nov. 30, 2001 (FR) 0115518

(51) Int. Cl. A61K 7/06; A61K 31/22; A61K 31/203; A61K 31/015; A61K 31/07; A61K 31/11

(52) U.S. Cl. 424/401; 514/559; 514/546; 514/703; 514/763; 514/725

(57) ABSTRACT

A method for preventing or combating chapping, tautness and/or redness comprising topical application onto skin or mucous membranes of a composition comprising, in a physiologically acceptable medium, preferably from 0.00001 to 0.1% of a retinoid and/or a carotenoid, and preferably from 0.001 to 1% of acexamic acid or a salt thereof is provided.

### FIELD OF THE INVENTION

The present invention relates to methods for preventing or combating chapping, tautness and/or redness comprising, inter alia, topical application onto skin or mucous membranes of a composition comprising, in a physiologically acceptable medium, from 0.00001 to 0.1% of a retinoid and/or a carotenoid, and from 0.001 to 1% of acexamic acid or a salt thereof. The present invention also relates to other uses of such a composition, for example, to promote the re-epithelialization of the skin or mucous membranes.

### BACKGROUND OF THE INVENTION

Environmental attacking factors, in particular variations in temperature and contact with irritant chemical substances (such as detergents), cause drying of the skin and mucous membranes, which is responsible for sensations of discomfort, tautness, itching, redness, or even chapping or cracks.

This is particularly true for the areas of the human body that are particularly exposed, and moreover fragile, i.e. the lips and the hands, since these areas have very thin skin. Moreover, these unpleasant sensations are more pronounced in the case of stressed individuals or the elderly, in whom the skin barrier is weakened, especially due to a reduction in epidermal biosyntheses during ageing.

The skin disorders resulting from these environmental attacking factors are due to an impairment, or even a break, in the continuity of the dermal covering by the epidermal cells normally forming a continuous and multilayered protective “carpet”.

The usefulness of having available a composition for reconstituting or preserving this “protective carpet”, i.e. of allowing the re-epithelialization of tissues, may thus be appreciated.

Such a composition would also be advantageous for people suffering from traumatic injuries such as extended wounds and bums. In the case of major bum sufferers, especially, a factor limiting the vital prognosis is the fact that a minimum area of skin must maintain, as best possible, the characteristics of “normal” skin as regards the number of epidermal cell layers and the integrity of the barrier function.

Various solutions have been envisaged hitherto for preventing or treating the skin disorders mentioned above.

Commercially available hand creams usually contain compounds for protecting the skin by forming a protective film thereon (petroleum jelly, collagen, waxes, plant oils or silicone oils), by moisturizing it (allantoin, urea, glycerol, sorbitol or ammonium lactate), by soothing it or promoting its cicatrization (aloe, bisabolol, vitamins A, E, F and B5, and allantoin), by screening out UVA and/or UVB radiation, and/or by nourishing it (essential fatty acids and karite butter).

However, the efficacy of these creams is occasionally insufficient and they especially have the drawback of giving a relatively uncosmetic greasy and sticky feel.

In addition, patent application EP-0 940 137 has already proposed the use of acexamic acid [also known as acetamidocaproic acid or 6-(acetylamino) hexanoic acid] in free form or in the form of the zinc salt, in a composition for protecting and caring for the lips, in particular against chapping. It has also been suggested in the publication by Guillard et al., Pharmacology 34: 296-300 (1987) to use acexamic acid in the treatment of wounds and to promote cicatrization. However, it has been demonstrated that, although acexamic acid does indeed allow the formation of a new epithelium in vitro, the epithelial layers formed are not of very good quality since they are fusiform and non-sealing.

It is moreover known that retinoids, including retinol, are capable of accelerating the re-epithelialization of injured skin by activating epidermal proliferation (M. Verschoore et al., Topical Retinoids—Their Uses in Dermatology, Dermatological Clinics, Vol. 11, No. 1, January 1993). However, it has been demonstrated that the epidermis thus obtained is not thick enough or sufficiently differentiated.

It was already known from CUCIUREANU et al., Caracterisation physico-chimique des onguents lipophiles avec vitamine A, Congr. Int. Technol. Pharm., Vol. 4, pp. 196-202 (1992) to introduce acexamic acid, as an anti-oxidant, into lipophilic preparations containing retinoids such as retinyl acetate, retinal or retinoic acid, in order to limit the degradation of these compounds. However, this document does not suggest that the preparation thus stabilized might have any re-epithelialization effect and only mentions the well-known use of retinoids against actinic aging.

There is thus still a need for a composition for cosmetic or dermatological use, for obtaining satisfactory re-epithelialization of the skin or mucous membranes, both quantitatively and qualitatively, while affording a cosmetically acceptable feel.

### SUMMARY OF THE INVENTION

It has now been discovered that the combined or successive use of acexamic acid (or a salt thereof) and at least one compound selected from the group consisting of a retinoid, a carotenoid and mixtures thereof allows this need to be satisfied.

### DETAILED DESCRIPTION OF THE INVENTION

According to the present invention, a method for preventing or combating chapping, tautness and/or redness comprising topical application onto skin or mucous membranes of a composition comprising, in a physiologically acceptable medium, preferably from 0.00001 to 0.1% of a retinoid and/or a carotenoid, and preferably from 0.001 to 1% of acexamic acid or a salt thereof is provided.

This composition is thus advantageously suitable for application to the hands, in particular dry and/or damaged hands. Such a composition may also be suitable for application to the lips.

The composition according to the present invention protects skin or mucous membranes against environmental attacking factors. It gives a long-lasting result without affording a greasy or sticky feel.

It has been discovered that the combined or sequential use of acexamic acid or a salt thereof and of a retinoid and/or a carotenoid has the advantage not only of allowing the potentiation via acexamic acid of the effect of the retinoid and/or carotenoid on the proliferation and differentiation of epithelial cells, but also of combating the long-term negative effects of acexamic acid on epithelial differentiation.

Any suitable retinoid can be used in accordance with the present invention. Preferably, the retinoids used in accordance with the present invention are selected from the group consisting of retinol, retinal, 13-cis-retinoic acid, all-trans-retinoic acid and retinyl esters such as retinyl acetate, retinyl propionate and retinyl palmitate. Retinyl palmitate is most preferred for use in the present invention.

Any suitable carotenoid can be used in accordance with the present invention. Preferably, the carotenoids used in accordance with the present invention are those possibly having vitamin A activity, some of which are retinoid precursors such as, for example, α-carotene, β-carotene, lycopene, zeaxanthin, lutein, ataxanthin, canthaxanthin and cryptoxanthin.

The carotenoid used according to the present invention may be of natural or synthetic origin. The term “natural origin” means the carotenoid in pure form or in solution, irrespective of its concentration in the solution, obtained from a natural element. The term “synthetic origin” means the carotenoid, in pure form or in solution, irrespective of its concentration in the solution, obtained by chemical synthesis.

When the carotenoid is of natural origin, it may be obtained from plant material derived from a whole plant grown in vivo or obtained from in vitro culturing. The expression “in vivo culturing” means any standard type of culturing, i.e. in soil, in the open air or in a greenhouse, or alternatively out of soil. The expression “in vitro culturing” means all the techniques known to those skilled in the art for artificially obtaining a plant or a plant part. The selection pressure imposed by the physicochemical conditions during the growing of the plant cells in vitro makes it possible to obtain a standardized plant material that is available throughout the year, in contrast with plants grown in vivo.

Preferably, according to the present invention, a plant obtained from in vivo culturing is used.

The carotenoids that are most preferred according to the present invention are carotene and lycopene.

Lycopene is a natural pigment found in ripe fruit, particularly in tomatoes. Its structure is similar to that of β-carotene. It may be in cis or trans form.

By way of example, a lycopene-rich tomato extract prepared by the company Metaphar, sold under the name LycOMato® and consisting of an oleoresin extract (fatty phase) containing 6% of pure lycopene, can be used according to the present invention.

Any suitable acexamic acid or salt thereof can be used in accordance with the present invention. Acexamic acid salts that may be mentioned include, for example, the organic and mineral salts preferably having an activating effect on urokinase (which is an enzyme involved, particularly in elderly or stressed individuals, in epidermal regeneration and also in the phenomena of re-epithelialization and cicatrization). Preferably, such salts include the alkaline-earth metal salts and the transition metal salts of acexamic acid, such as, for example, the zinc, calcium and/or magnesium salts of acexamic acid.

Acexamic acid is preferably used in free form, more preferably in the form of powder, sold by the company Sanofi Chimie under the trade name Acide Acexamique N.

The composition according to the invention generally contains an amount of retinoid or carotenoid and of acexamic acid that is effective to obtain the desired effect. Preferably, this amount is from 0.001% to 1% by weight, more preferably from 0.01% to 0.1% by weight, of acexamic acid and/or a salt thereof, and preferably from 0.00001% to 0.1% by weight, more preferably from 0.001% to 0.01% by weight, of retinoid or carotenoid, relative to the total weight of the composition.

The composition according to the present invention is advantageously suitable for topical application to the skin or mucous membranes. The term “skin” means both facial skin and body skin, in particular the skin of the hands. The term “mucous membranes” designates any mucous membrane material, preferably the lips. The expression “physiologically acceptable medium” means a medium that is compatible with the skin and/or mucous membranes.

Besides its primarily cosmetic uses, the combination of acexamic acid and of retinoid or carotenoid according to the present invention is also advantageous in the field of dermatology. For example, in this field, it will be advantageous to use at least one first compound chosen from acexamic acid and salts thereof, and of at least one second compound chosen from retinoids and carotenoids, to manufacture, respectively, a first and a second dermatological preparation intended to be administered successively to promote the re-epithelialization of the skin or mucous membranes.

Another object of the present invention is thus the use of at least one first compound chosen from acexamic acid and salts thereof, and of at least one second compound chosen from retinoids and carotenoids, to manufacture, respectively, a first and a second dermatological preparation intended to be administered successively to promote the re-epithelialization of the skin or mucous membranes.

Preferably these first and second preparations are used to promote the cicatrization of wounds or bums.

According to one embodiment of the present invention, the first preparation is applied topically to the skin or mucous membranes and the second preparation is administered orally or parenterally. According to another embodiment, the first preparation and the second preparation are applied topically to the skin or mucous membranes. According to yet another embodiment, the first and second preparations may be administered orally.

The successive administration of the two compounds according to the present invention may, according to another embodiment, be used in the manufacture of a reconstructed or artificial skin.

The invention thus also extends its scope to a process for manufacturing a reconstructed or artificial skin, comprising (a) applying a first composition comprising at least one first compound chosen from acexamic acid and salts thereof to a dermal equivalent, (b) applying a second composition comprising at least one second compound chosen from retinoids and carotenoids to the dermal equivalent.

The expression “dermal equivalent” means any support of biological origin (i.e. of cellular origin, preferably comprising fibroblasts and/or endothelial cells) and/or biochemical origin (i.e. of protein origin, preferably comprising collagen and/or fibronectin, and/or based on mucopolysaccharides, preferably comprising hyaluronic acid), the support also possibly comprising a synthetic portion.

The composition and the preparations according to the present invention may be in any presentation form normally used in cosmetics and/or dermatology. Preferably, they are in the form of an optionally gelled oily solution, an emulsion obtained by dispersing a fatty phase in an aqueous phase (O/W) or, conversely, (W/O), a triple emulsion (W/O/W or O/W/O), a vesicular dispersion of ionic type (liposomes or oleosomes) and/or non-ionic type (niosomes) and/or a dispersion of nanocapsules or nanospheres.

The composition according to the present invention is most preferably in the form of a water-in oil (W/O) emulsion.

In accordance with preferred embodiments, the retinoid or carotenoid used in the composition according to the present invention is encapsulated, most preferably in nanocapsules, so as to delay its release and thus to obtain a sequential effect of the acexamic acid and then of the retinoid and/or carotenoid after applying the composition to the skin. It has been demonstrated, for example in Example 1 below, that the sequential application of these compounds allows optimum re-epithelialization of the skin.

The composition and the preparations according to the present invention may be in any acceptable form. For example, they may be more or less fluid and may have the appearance of a white or coloured cream, an ointment, a milk, a lotion, a serum, a paste or a mousse. They may be applied to the skin in the form of an aerosol or a patch. They may also be in solid form, in particular in the form of a pencil for the lips or a lipstick. They may be used as a care product and/or as a makeup product for the skin or the lips and/or as a shaving product.

The composition and the preparations according to the present invention may also contain adjuvants that are commonly used in cosmetics and dermatology such as, for example, hydrophilic or lipophilic gelling agents, hydrophilic or lipophilic active agents,

preserving agents, antioxidants, solvents, fragrances, fillers, screening agents, pigments, odour absorbers and dyestuffs. The amounts of these various adjuvants are those conventionally used in the field under consideration, for example from 0.01% to 200 of the total weight of the composition. Depending on their nature, these adjuvants may be introduced into the fatty phase or into the aqueous phase. In any case, these adjuvants, and the proportions thereof, will be chosen so as not to harm the desired properties of the combination of active agents according to the present invention.

When the composition according to the invention is an emulsion, the proportion of the fatty phase can range from 5% to 80% by weight, preferably from 5% to 50% by weight, relative to the total weight of the composition. The oils, emulsifiers and co-emulsifiers used in the composition in emulsion form are chosen from those conventionally used in the field in question. The emulsifier and the co-emulsifier are generally present in the composition in a proportion ranging from 0.3% to 30% by weight, preferably from 0.5% to 20% by weight, relative to the total weight of the composition.

Suitable oils for use in the composition of the present invention include, for example:

hydrocarbon-based oils of animal origin, such as perhydrosqualene;

hydrocarbon-based oils of plant origin, such as liquid triglycerides of fatty acids containing from 4 to 10 carbon atoms and the liquid fraction of karite butter;

synthetic esters and synthetic ethers, especially of fatty acids, for instance oils of formulae R¹COOR² and R¹0R² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, and hydrogenated polyisobutene such as parleam oil;

fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluoro oils, for instance those described in document JP-A-2 295 912;

silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones) such as cyclohexasiloxane; polydimethylsiloxanes comprising alkyl, alkoxy or phenyl groups, that are pendent or at the end of a silicone chain, these groups containing from 2 to 24 carbon atoms; phenylsilicones, for instance phenyltrimethicones, phenyldimethicones, phenyltri-methylsiloxydiphenylsiloxanes, diphenyldimethicones, diphenylmethyldiphenyltrisiloxanes, 2-phenylethyl-trimethylsiloxysilicates and polymethylphenylsiloxanes;

mixtures thereof.

Suitable emulsifiers and co-emulsifiers that may be used in accordance with the present invention include, for example, O/W emulsifiers such as fatty acid esters of polyethylene glycol, especially PEG-100 stearate, and fatty acid esters of glycerol, such as glyceryl stearate, and W/O emulsifiers such as the oxyethylenated poly(methylcetyl)(dimethyl)methyl-siloxane sold under the trade name ABIL WE09 from the company Degussa Goldschmidt, or the mixture of acetyl ethylene glycol stearate and of glyceryl tristearate sold by the company Guardian under the trade name UNITWIX.

Suitable hydrophilic gelling agents include, for example, carboxyvinyl polymers (carbomer), acrylic copolymers such as acrylate/alkylacrylate copolymers, polyacrylamides, polysaccharides, natural gums and clays, and lipophilic gelling agents such as, for example, modified clays, for example, bentones, metal salts of fatty acids, hydrophobic silica and polyethylenes.

Suitable fillers that may be used in the composition of the present invention include, for example, pigments; silica powder; talc; starch crosslinked with octenylsuccinic anhydride sold by the company National Starch under the name DRY FLO PLUS (28-1160); polyamide particles and especially those sold under the name ORGASOL by the company Atochem; polyethylene powders; microspheres based on acrylic copolymers, such as those made of ethylene glycol dimethacrylate/lauryl methacrylate copolymer sold by the company Dow Corning under the name POLYTRAP; expanded powders such as hollow microspheres and especially the microspheres sold under the name EXPANCEL by the company Kemanord Plast or under the name MICROPEARL F 80 ED by the company Matsumoto; silicone resin microbeads such as those sold under the name TOSPEARL by the company Toshiba Silicone; and mixtures thereof. These fillers may be present in amounts ranging from 0 to 20% by weight, preferably from 1% to 10% by weight, relative to the total weight of the composition or of the preparation according to the present invention.

Finally, examples of active agents that may be used in the composition or the preparations according to the present invention include, for example, collagen, allantoin, urea, glycerol, sorbitol, ammonium lactate, aloe extracts, bisabolol, and vitamins E, F and B5. In the event of incompatibility or to stabilize them, at least some of the active agents mentioned above may be incorporated into spherules, especially ionic or non-ionic vesicles and/or nanoparticles (nanocapsules and/or nanospheres).

UVA and/or UVB screening agents chosen from organic screening agents and mineral screening agents optionally coated to make them hydrophobic may also be introduced into the composition or the preparations according to the present invention.

According to one preferred embodiment of the present invention, the acexamic acid may be compatibilized in the fatty phase of the compositions or preparations according to the present invention, in particular when an anhydrous phase is involved, using at least one polyol that is liquid at room temperature, with an IOB value of between 1 and 7. Specifically, it has been discovered that these polyols are very good solvents for acexamic acid.

The IOB parameter is known to those skilled in the art from a certain number of publications, for instance the article by A. Fujita Pharm. Bull. 2, 163 173 (1954) and documents J09/151,109, J08/217,639 or J09/175,925.

Examples of polyols that may be used in the compositions and preparations of the present invention include, for example, alkylene glycols, such as propylene glycol, butylene glycol, pentylene glycol and hexylene glycol; polyethylene glycols (PEG) containing from 4 to 8 ethylene glycol units, such as PEG-4, PEG-6 or PEG-8; glycerol; and panthenol.

Where the first and/or second preparation is (are) administered orally, it (they) may be in any presentation form that is suitable for this mode of administration, for example in the form of splitable or non-splitable tablets, granules, wafer capsules, gel capsules, solutes, suspensions or solutions comprising a suitable excipient. Examples of excipients that are suitable for this mode of administration comprise a mixture of dextrose and cellulose optionally reinforced with magnesium stearate as binder and lubricant.

When the first preparation is administered orally, it contains an amount of acexamic acid or of a salt thereof that is sufficient to obtain the desired effect. Preferably, this is an amount that allows the administration, in one or more dosage intakes, of from 300 to 600 mg of acexamic acid per day.

When the second preparation is administered orally, it contains an amount of retinoid or carotenoid that is sufficient to obtain the desired effect. Preferably, this is an amount providing a dose of carotene corresponding to 400 μg of vitamin A, i.e. about 3 mg of carotene, or providing a dose of other carotenoids such as lycopene, zeaxanthin, cryptoxanthin or lutein, corresponding to 1.6 mg per day.

The present invention will be understood more clearly, and its advantages will emerge more clearly, in the light of the following examples, which are given for illustrative purposes and with no limitation.

### EXAMPLES

### Example 1

### Test of Re-Epithelialization and Epithelial Proliferation

a) Materials and Method

Reconstitutions of dermal equivalent were prepared by successively mixing together, in a Petri dish 6 cm in diameter, the following components:

a culture medium (MEM-Dulbecco concentrated 1.76 times),

type I collagen dissolved in 0.1% acetic acid, neutralized with sodium hydroxide (5 mM),

10% defatted foetal calf serum, and

2.5×10⁵ fibroblasts suspended in MEM medium containing 10% foetal calf serum.

The dermal equivalent was formed by polymerization in an oven at 37° C., under an atmosphere of 5% carbon dioxide.

Certain lattices were pretreated for three days, while others underwent no treatment. On the third day, a 2 mm fragment of cutaneous epithelium was implanted onto the dermal equivalents (5 series were prepared).

The lattices obtained before epidermization and the skin equivalents thus reconstituted were treated as indicated in Table 1 below:

TABLE 1

| | Treatment in the lattice | Treatment of the |
|---|---|---|
| Condition | preparation medium | reconstituted skin |
| 1 | none | none |
| 2 | none | Composition B at 1% (v/v) |
| 3 | Composition A at 1% (v/v) | none |
| 4 | Composition A at 1% (v/v) | Composition B at 1% (v/v) |
| 5 | Composition A at 1% (v/v) | Composition A at 1% (v/v) |

Composition A consisted of 4% by weight of acexamic acid in water.

Composition B consisted of a 30% by weight dispersion of nanocapsules, containing 2.5% by weight of retinyl palmitate in 70% by weight of water.

The medium was changed every two days for 20 days. The reconstructed skins were removed on the twentieth day and tested as described below.

b) Histological Study

A histological study of the epidermization of the reconstituted skins was performed after staining sections with haematoxylin.

The number of cell layers in the newly formed epithelium (from 0 to 5 layers) was measured. The fusiform or cubic aspect of the cells, especially in the basal layer, was specified, as was the presence or absence of differentiation of the corpus mucosa.

Better histological quality of condition 4 over the other conditions was thus demonstrated. This is reflected by the formation of a very thick newly formed epithelium (from 1 to 5 cell layers) associated with good differentiation and the presence of a layer of basal cells of cubic and generally sealing aspect.

In contrast, conditions 1, 3 and 5, which were not treated with composition B, show cells that are usually sealing, but of fusiform aspect, whereas condition 2 shows a lower number of layers than condition 4.

c) Immunohistochemical Study

5-Bromo-2′-deoxyuridine (BrDU), a marker of 5 cells in the S phase of the cell cycle, was added to the culture medium of the reconstructed skins between the eighth and twenty-fifth days, for two hours, in order to analyse the epithelial proliferation.

This analysis was completed by an immunohistochemical study of the conditions, which consists in analysing the proliferation after immunolabelling, using an anti-Ki67 antibody, in order to measure all the cells undergoing proliferation rather than only those in the phase of DNA synthesis.

After fixing in formaldehyde and inclusion in paraffin, the immunodetection of the cells in the proliferation phase was performed using a two-layer indirect immunoperoxidase technique and revealed in DAB (Amersham kit).

Counting of the number of epithelium-labelled cells was carried out. A percentage of cells in mitosis was thus calculated.

The results are given in Table 2 below.

TABLE 2

| | % of positive basal |
|---|---|
| Condition | cells |
| 1 | 7.8 ± 3.05 |
| 2 | 24.2 ± 8 |
| 3 | 9.5 ± 6.3 |
| 4 | 23.3 ± 5.5 |
| 5 | 20.3 ± 4.9 |

It follows from this table that condition 4 allows the production of a very satisfactory level of proliferation.

d) Conclusion

Comparison of the results obtained below for the various test conditions shows that condition 4, which corresponds to a skin sample pretreated (before epidermization) with acexamic acid and treated (after formation of the reconstituted skin) with retinyl palmitate, gives the best results, whether in terms of modifying the proliferation (quantitative evaluation) or modifying the epithelialization (qualitative evaluation).

This test thus demonstrates the advantage of this type of sequential treatment in the phenomenon of re-epithelialization.

### Example 2

### Test of Re-Epithelialization and of Epithelial Proliferation

A test similar to that described in Example 1 was performed, except that the reconstituted skins were treated after epidermization only, by introducing into the culture medium, on the seventh day, a composition consisting, in weight percentages: of 4% acexamic acid; of 30% of nanocapsules containing 3.5% by weight of retinyl palmitate; of 0.075% preserving agent and of 0.075% antioxidant in water.

The histological analysis shows that, on the twenty-fifth day, the five series of samples produced all show a neo-epithelium formed of a basal layer and of one to four layers of corpus mucosa. By comparison, the newly formed epithelium of the same untreated reconstituted skins comprises only one basal layer and appears with a delay of five to ten days compared with the treated series.

The immunohistochemical analysis reveals considerable cell division, corresponding to the proliferation of the cells, whereas the cellular proliferation phase of the untreated reconstructed skins appears, in this case also, with a delay.

This test thus demonstrates, compared with the untreated control, a positive modification of the re-epithelialization by the combination of acexamic acid with retinyl palmitate, accompanied by a reduction in the time to reconstitute the epithelia correlated with an increase in the number of epithelial cells in mitosis.

### Example 3

### Cosmetic Composition

| | | |
|---|---|---|
| Octyldodecanol | | 4% |
| Magnesium sulphate | | 0.7% |
| Mineral oil | | 18% |
| Preserving agents | | 0.75% |
| Glycerol | | 7% |
| Cetyl dimethicone copolyol | | 2% |
| Retinyl palmitate nanocapsules | | 0.3% |
| (2.5% active material) | | |
| Acexamic acid | | 0.04% |
| Water | qs | 100% |

This cream may be applied to the hands as often as necessary to soothe redness and tautness and to reclose chapping.

### Example 4

### Cosmetic Composition

The composition below is prepared in a manner conventional to those skilled in the art:

| | | |
|---|---|---|
| Octyldodecanol | | 4% |
| Magnesium sulphate | | 0.7% |
| Mineral oil | | 18% |
| Preserving agents | | 0.75% |
| Glycerol | | 7% |
| Cetyl dimethicone copolyol | | 2% |
| Retinyl palmitate nanocapsules | | 0.3% |
| (2.5% active material) | | |
| Lycopene | | 0.0013% |
| Acexamic acid | | 0.04% |
| Water | qs | 100% |

### Example 5

### Preparations for Sequential Administration

Compositions A and B below are prepared, in a manner conventional to those skilled in the art.

Composition A: skin cream

| | | |
|---|---|---|
| Octyldodecanol | | 4% |
| Magnesium sulphate | | 0.7% |
| Mineral oil | | 18% |
| Preserving agents | | 0.75% |
| Glycerol | | 7% |
| Cetyl dimethicone copolyol | | 2% |
| Retinyl palmitate nanocapsules | | 0.3% |
| (2.5% active material) | | |
| Acexamic acid | | 0.04% |
| Water | qs | 100% |

Composition B: gel capsules

| | |
|---|---|
| beta-Carotene | 5 mg |
| Lycopene | 2.6 mg |
| Maltodextrin | 300 mg |

The skin composition is intended to be applied in the morning to the exposed parts of the body, while the gel capsules will be ingested at the end of the day. This protocol will be repeated for several days, or even weeks.

### Example 6

### Preparations for Sequential Administration

Compositions A and B below are prepared, in a manner conventional to those skilled in the art.

Composition A: skin cream

| | | |
|---|---|---|
| Octyldodecanol | | 4% |
| Magnesium sulphate | | 0.7% |
| Mineral oil | | 18% |
| Preserving agents | | 0.75% |
| Glycerol | | 7% |
| Cetyl dimethicone copolyol | | 2% |
| Retinyl palmitate nanocapsules | | 0.3% |
| (2.5% active material) | | |
| Acexamic acid | | 0.04% |
| Water | qs | 100% |

Composition B: nutritional supplement - soft capsules

| | |
|---|---|
| beta-Carotene | 5 mg |
| Lycopene | 2.6 mg |
| Hydrogenated soybean oil | 40 mg |
| Wheat oil | 95 mg |
| Soybean lecithin | 20 mg |
| Natural tocopherols | 5 mg |
| Ascorbic acid | 30 mg |

The above skin composition is intended to be applied to the exposed parts of the body substantially simultaneously with the ingestion of the nutritional supplement, the use of which may be prolonged for several days. Since the kinetics of absorption of the nutrients are slower than the percutaneous absorption of acexamic acid, a sequential effect of the acexamic acid and the carotenoids will thus be obtained, which affords maximum benefit to the skin.

The present application claims priority from French patent application no. 0115518 filed Nov. 30, 2001, the entire contents of which is hereby incorporated by reference.

1. A method for promoting re-epithelialization of the skin or mucous membranes comprising providing a re-epithelialization promoting effective amount of (1) acexamic acid or a salt thereof; and (2) a retinoid and/or a carotenoid to the skin or mucous membranes.

2. The method according to claim 1, wherein the retinoid is selected from the group consisting of retinol, retinal, 13-cis-retinoic acid, all-trans retinoic acid and retinyl esters.

3. The method according to claim 2, wherein the retinyl esters are selected from the group consisting of retinyl acetate, retinyl propionate and retinyl palmitate.

4. The method according to claim 2, wherein the retinoid is retinol or retinyl palmitate.

5. The method according to claim 1, wherein the carotenoid is selected from the group consisting of α-carotene, β-carotene, lycopene, zeaxanthin, lutein, ataxanthin, canthaxanthin and cryptoxanthin.

6. The method according to claim 1, wherein the retinoid and/or carotenoid is encapsulated.

7. The method according to claim 1, wherein acexamic acid is provided to skin or mucous membranes.

8. The method according to claim 1, wherein the acexamic acid salts are selected from the group consisting of the alkaline-earth metal salts and the transition metal salts of acexamic acid.

9. The method according to claim 8, wherein the acexamic acid salts are selected from the group consisting of zinc, calcium and magnesium salts of acexamic acid.

10. The method according to claim 1, wherein the (1) acexamic acid or salt thereof; and (2) retinoid and/or carotenoid are in the same composition.

11. The method according to claim 10, wherein the composition comprises from 0.001% to 0.1% by weight of acexamic acid or a salt thereof, relative to the total weight of the composition.

12. The method according to claim 10, wherein the composition comprises from 0.01% to 0.1% by weight of acexamic acid or salt thereof, relative to the total weight of the composition.

13. The method according to claim 10, wherein the composition comprises from 0.00001% to 0.1% by weight of retinoid or carotenoid, relative to the total weight of the composition.

14. The method according to claim 10, wherein the composition comprises from 0.001% to 0.1% by weight of retinoid or carotenoid, relative to the total weight of the composition.

15. The method according to claim 10, wherein the composition is in the form of a water-in-oil emulsion.

16. The method according to claim 10, wherein the composition is applied to the skin of the hands.

17. The method according to claim 10, wherein the composition is applied to the lips.

18. The method according to claim 1, wherein cicatrization of wounds or burns is promoted.

19. The method according to claim 1, wherein the acexamic acid or salt thereof is applied topically to the skin or mucous membranes and the retinoid and/or carotenoid is administered orally or by injection.

20. The method according to claim 1, wherein both the acexamic acid or salt thereof and the retinoid and/or carotenoid are administered topically.

21. A method for manufacturing a reconstructed or artificial skin, comprising (a) applying a first composition comprising at least one acexamic acid or salt thereof to a dermal equivalent, and (b) applying a second composition comprising at least one retinoid and/or carotenoid to the dermal equivalent.

22. A method for preventing or combating chapping, tautness and/or redness of skin or mucous membranes comprising providing a chapping, tautness and/or redness preventing or combating effective amount of (1) acexamic acid or a salt thereof; and (2) a retinoid and/or a carotenoid to the skin or mucous membranes.

23. The method according to claim 22, wherein the retinoid is selected from the group consisting of retinol, retinal, 13-cis-retinoic acid, all-trans retinoic acid and retinyl esters.

24. The method according to claim 23, wherein the retinyl esters are selected from the group consisting of retinyl acetate, retinyl propionate and retinyl palmitate.

25. The method according to claim 23, wherein the retinoid is retinol or retinyl palmitate.

26. The method according to claim 22, wherein the carotenoid is selected from the group consisting of α-carotene, β-carotene, lycopene, zeaxanthin, lutein, ataxanthin, canthaxanthin and cryptoxanthin.

27. The method according to claim 22, wherein the retinoid and/or carotenoid is encapsulated.

28. The method according to claim 22, wherein acexamic acid is provided to skin or mucous membranes.

29. The method according to claim 22, wherein the acexamic acid salts are selected from the group consisting of the alkaline-earth metal salts and the transition metal salts of acexamic acid.

30. The method according to claim 29, wherein the acexamic acid salts are selected from the group consisting of zinc, calcium and magnesium salts of acexamic acid.

31. The method according to claim 22, wherein the (1) acexamic acid or salt thereof; and (2) retinoid and/or carotenoid are in the same composition.

32. The method according to claim 31, wherein the composition comprises from 0.001% to 0.1% by weight of acexamic acid or salt thereof, relative to the total weight of the composition.

33. The method according to claim 31, wherein the composition comprises from 0.01% to 0.1% by weight of acexamic acid or salt thereof, relative to the total weight of the composition.

34. The method according to claim 31, wherein the composition comprises from 0.00001% to 0.1% by weight of retinoid or carotenoid, relative to the total weight of the composition.

35. The method according to claim 31, wherein the composition comprises from 0.001% to 0.1% by weight of retinoid or carotenoid, relative to the total weight of the composition.

36. The method according to claim 31, wherein the composition is in the form of a water-in-oil emulsion.

37. The method according to claim 31, wherein the composition is applied to the skin of the hands.

38. The method according to claim 31, wherein the composition is applied to the lips.

39. The method according to claim 22, wherein cicatrization of wounds or bums is promoted.

40. The method according to claim 22, wherein the acexamic acid or salt thereof is applied topically to the skin or mucous membranes and the retinoid and/or carotenoid is administered orally or by injection.

41. The method according to claim 22, wherein both the acexamic acid or salt thereof and the retinoid and/or carotenoid are administered topically.
